(19) United States
(12) Patent Application Publication
(10) Pub. No.: US 2020/0275900 A1
(43) Pub. Date: Sep. 3, 2020

(54) DIAGNOSTIC IMAGING APPARATUS

(71) Applicant: Shimadzu Corporation, Kyoto (JP)

(72) Inventors: Kazushige TACHIBANA, Kyoto (JP); Atsushi OHTANI, Kyoto (JP); Tetsuro MIZUTA, Kyoto (JP)

(73) Assignee: Shimadzu Corporation, Kyoto (JP)

(21) Appl. No.: 16/289,767

(22) Filed: Mar. 1, 2019

(51) Int. Cl. A61B 6/00; G01T 1/29; A61B 6/03; A61B 6/04

(52) U.S. Cl. CPC A61B 6/502; G01T 1/2985; A61B 6/501; A61B 6/4452; A61B 6/0435; A61B 6/037

(57) ABSTRACT

A diagnostic imaging apparatus includes an imaging unit including an imager that images a target to be imaged, and a rotation mechanism that rotates the imaging unit to switch the imaging unit to a first state in which a first target area to be imaged of the target is imaged and a second state in which a second target area to be imaged of the target different from the first target area to be imaged is imaged.

### CROSS-REFERENCE TO RELATED APPLICATIONS

The related application number 2016-163725, Diagnostic Imaging Apparatus, Aug. 24, 2016, Kazushige Tachibana, Atsushi Ohtani, and Tetsuro Mizuta, upon which this patent application is based, is hereby incorporated by reference.

### FIELD

The present invention relates to a diagnostic imaging apparatus.

### BACKGROUND

A diagnostic imaging apparatus including an imager that images a target to be imaged is known in general, as disclosed in International Publication No. 2011/125212, for example.

International Publication No. 2011/125212 discloses a PET apparatus that includes a whole-body PET (Positron Emission Tomography) apparatus including a whole-body PET detector that detects annihilation radiations, and a part-specific PET apparatus including a part-specific (head or breast) PET detector that detects annihilation radiations. In this PET apparatus, the part-specific PET apparatus corresponding to a specific part that requires detailed imaging is provided in the whole-body PET apparatus such that it is possible to image a wide range while imaging a specific part in detail.

However, in the PET apparatus described in International Publication No. 2011/125212, in order to image different specific parts in detail, separate part-specific PET apparatuses corresponding to imaging of the respective specific parts are required. Therefore, the number of components disadvantageously increases, and the apparatus structure disadvantageously becomes complex.

### SUMMARY

The present invention has been proposed in order to solve the aforementioned problems, and an object of the present invention is to provide a diagnostic imaging apparatus in which an increase in the number of components and the complex apparatus structure can be significantly reduced or prevented.

In order to attain the aforementioned object, a diagnostic imaging apparatus according to an aspect of the present invention includes an imaging unit including an imager that images a target to be imaged, and a rotation mechanism that rotates the imaging unit to switch the imaging unit to a first state in which a first target area to be imaged of the target is imaged and a second state in which a second target area to be imaged of the target different from the first target area to be imaged is imaged.

As described above, the diagnostic imaging apparatus according to this aspect of the present invention includes the rotation mechanism that rotates the imaging unit to switch the imaging unit to the first state in which the first target area to be imaged of the target is imaged and the second state in which the second target area to be imaged of the target different from the first target area to be imaged is imaged. Accordingly, the imaging unit is rotated by the rotation mechanism such that the imaging unit can be switched to states in which different target areas to be imaged are imaged, and thus it is not necessary to provide a dedicated imaging unit for each target area to be imaged. Consequently, in the diagnostic imaging apparatus, an increase in the number of components and the complex apparatus structure can be significantly reduced or prevented.

In the aforementioned imaging apparatus according to this aspect, the imaging unit preferably further includes an imaging region in which the first target area to be imaged is placed in the first state and the second target area to be imaged is placed in the second state, and the imager preferably surrounds the imaging region. According to this structure, the first target area to be imaged or the second target area to be imaged placed in the imaging region can be reliably imaged by the imager that surround the imaging region.

The aforementioned diagnostic imaging apparatus according to this aspect preferably further includes a bed on which a human body as the target to be imaged is placed in a recumbent position, and the rotation mechanism preferably includes a rotary shaft that extends, in the imaging unit, in a short-side direction orthogonal to a longitudinal direction of the bed and about which the imaging unit is rotated. According to this structure, as compared with the case in which the rotary shaft extends in the longitudinal direction, an increase in the sizes of the rotary shaft and the rotation mechanism can be significantly reduced or prevented, and thus an increase in the size of the diagnostic imaging apparatus can be further significantly reduced or prevented.

In the aforementioned structure in which the rotation mechanism includes the rotary shaft, the imaging unit preferably further includes an imaging region in which a head of the human body in the recumbent position as the first target area to be imaged is placed in the first state and a breast of the human body in the recumbent position as the second target area to be imaged is placed in the second state, and the rotary shaft is preferably provided in a vicinity of an opening of the imaging region on a side opposite to the bed in the imaging unit in the first state. According to this structure, the rotary shaft is provided in the vicinity of the opening of the imaging region on the side opposite to the bed such that approach of the rotation range of the imaging unit to the bed can be significantly reduced or prevented, and thus inhibition of rotation of the imaging unit by the bed can be significantly reduced or prevented. Furthermore, the rotary shaft is provided in the vicinity of the opening of the imaging region such that the imaging region can be located within the narrow rotation range around the rotary shaft, and thus spacing apart of the imaging region from the bed can be significantly reduced or prevented.

In the aforementioned structure in which the rotation mechanism includes the rotary shaft, the imaging unit preferably further includes an imaging region in which a head of the human body in the recumbent position as the first target area to be imaged is placed in the first state and a breast of the human body in the recumbent position as the second target area to be imaged is placed in the second state, and the rotary shaft is preferably provided in a vicinity of an opening of the imaging region on the bed side in the imaging unit in the first state. According to this structure, the rotary shaft is provided in the vicinity of the opening of the imaging region on the bed side such that protrusion of a portion that supports the rotary shaft of the rotation mechanism to the side of the diagnostic imaging apparatus opposite to the bed can be significantly reduced or prevented, and thus an increase in the size of the diagnostic imaging apparatus can be significantly reduced or prevented. Furthermore, the rotary shaft is provided in the vicinity of the opening of the imaging region such that the imaging region can be located within the narrow rotation range around the rotary shaft, and thus spacing apart of the imaging region from the bed can be significantly reduced or prevented.

In the aforementioned structure in which the rotary shaft is provided in the vicinity of the opening of the imaging region, the rotary shaft is preferably provided at substantially a same height as that of an upper surface of the bed. According to this structure, the rotary shaft is provided at substantially the same height as that of the upper surface of the bed such that spacing apart of the imaging region from the upper surface of the bed can be significantly reduced or prevented as compared with the case in which the rotary shaft is provided at a height spaced apart from the upper surface of the bed.

The aforementioned structure further including the bed preferably further includes a headrest detachably attached to an end of the bed on the imaging unit side and that supports a head of the human body. According to this structure, when the headrest is attached to the end of the bed on the imaging unit side, the position of the headrest is adjusted such that the head of the recumbent human body can be placed at an appropriate position in the imaging region. Consequently, when the head of the recumbent human body is imaged as the target area to be imaged, the head of the recumbent human body can be easily imaged by the imager. Furthermore, the headrest is detached from the end of the bed on the imaging unit side such that it is possible to prevent the headrest from interfering with rotation of the imaging unit.

In the aforementioned structure further including the bed, the imaging unit preferably includes a support that extends in a direction away from the bed in the second state and supports the human body. Here, when the vicinity of the center of the human body such as the chest is imaged as the second target area to be imaged, a portion of the human body is located in a direction away from the bed relative to the imaging region of the imaging unit. Therefore, in the present invention, the support that extends in the direction away from the bed and supports the human body is provided in the imaging unit in the second state such that when the vicinity of the center of the human body is imaged as the second target area to be imaged, the recumbent human body can be supported by the support that extends in the direction away from the bed. Consequently, the human body can be securely kept in a recumbent position, and thus the second target area to be imaged can be stably imaged.

In the aforementioned diagnostic imaging apparatus according to this aspect, the first target area to be imaged is preferably a head of the human body in a supine position, the imager preferably surrounds the head of the human body within a vertical plane in the first state, the second target area to be imaged is preferably a breast of the human body in a prone position, and the imager preferably surrounds the breast of the human body within a horizontal plane in the second state. According to this structure, the head of the supine human body can be stably and reliably imaged. Furthermore, the breast of the human body can hang downward in a prone position, and thus a wide range of the breast of the human body can be stably and reliably imaged.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a schematic sectional view showing the head imaging state of a PET apparatus according to a first embodiment.

FIG. 2 is a top view showing the head imaging state of the PET apparatus according to the first embodiment.

FIG. 3 is a side view showing the head imaging state of the PET apparatus according to the first embodiment, as viewed from the imaging unit side.

FIG. 4 is a block diagram of the PET apparatus according to the first embodiment.

FIG. 5 is a schematic sectional view showing the breast imaging state of the PET apparatus according to the first embodiment.

FIG. 6 is a top view showing the breast imaging state of the PET apparatus according to the first embodiment.

FIG. 7 is a side view showing the breast imaging state of the PET apparatus according to the first embodiment, as viewed from the imaging unit side.

FIG. 8 is a diagram illustrating the rotation operation of an imaging unit of the PET apparatus according to the first embodiment.

FIG. 9 is a diagram showing a state during rotation in the rotation operation of the imaging unit of the PET apparatus according to the first embodiment.

FIG. 10 is a diagram illustrating the rotation operation of the imaging unit of the PET apparatus according to the first embodiment.

FIG. 11 is a schematic sectional view showing the head imaging state of a PET apparatus according to a second embodiment.

FIG. 12 is a schematic sectional view showing the breast imaging state of the PET apparatus according to the second embodiment.

FIG. 13 is a schematic sectional view showing the chest imaging state of an X-ray imaging apparatus according to a third embodiment.

FIG. 14 is a schematic sectional view showing the breast imaging state of the X-ray imaging apparatus according to the third embodiment.

### DETAILED DESCRIPTION

Embodiments of the present invention are hereinafter described with reference to the drawings.

### First Embodiment

### (Outline of PET Apparatus)

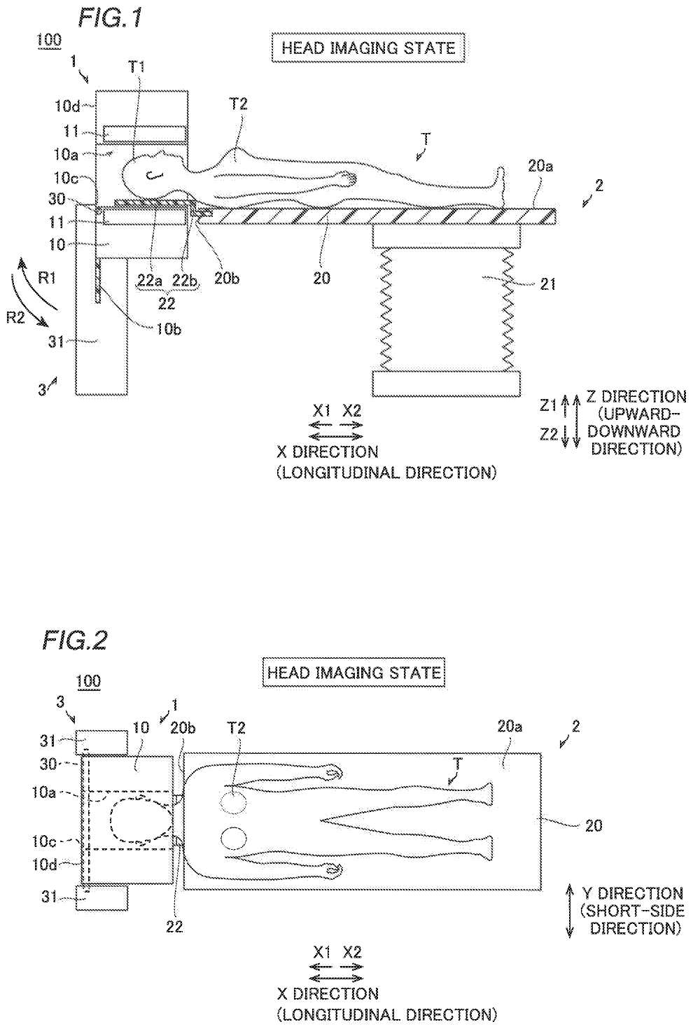

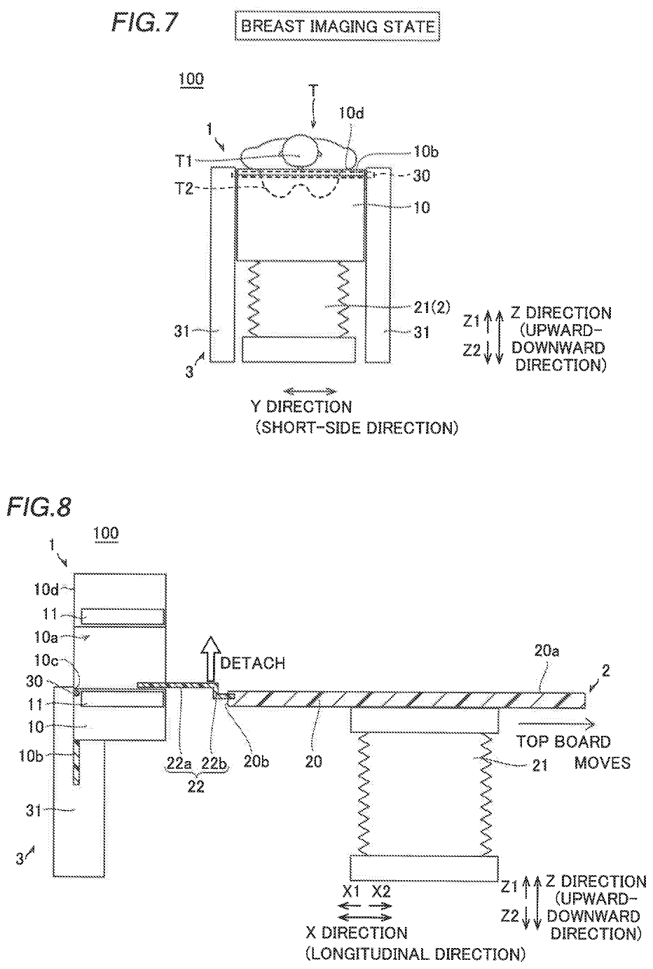

The structure of a PET apparatus 100 according to a first embodiment of the present invention is now described with reference to FIGS. 1 to 7. The PET apparatus 100 is an example of a “diagnostic imaging apparatus” in the claims.

The PET apparatus 100 is an apparatus that captures an image inside a human body T, using a drug labeled with a positron emitting nuclide. Specifically, the PET apparatus 100 acquires a position at which pair annihilation of the drug occurs by detecting a pair of gamma rays (radiations) generated by the pair annihilation of electrons and positrons of the drug. Furthermore, the PET apparatus 100 forms (captures) the image inside the human body T by acquiring a plurality of positions at which the pair annihilation of the drug occurs. The formed image is used in image diagnosis for determining the presence or absence of cancer cells, for example. The human body T is an example of a “target to be imaged” in the claims.

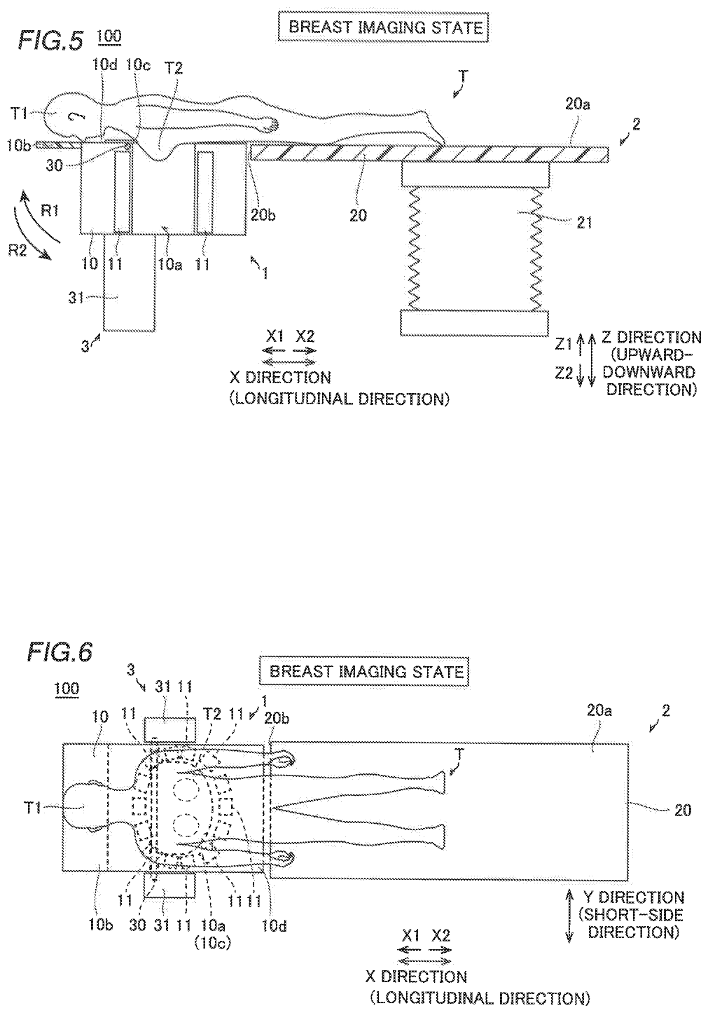

In the first embodiment, the PET apparatus 100 can switch between a head imaging state of imaging the head T1 of the supine human body T and a breast imaging state of imaging the breast T2 of the prone human body T. In the first embodiment, the structure of the PET apparatus 100 in the head imaging state shown in FIGS. 1 to 3 is described first, and then points of the breast imaging state of the PET apparatus 100 shown in FIGS. 5 to 7 different from the head imaging state are described. The head T1 and the breast T2 are examples of a “first target area to be imaged” and a “second target area to be imaged” in the claims, respectively. In addition, the head imaging state and the breast imaging state are examples of a “first state” and a “second state” in the claims, respectively.

### (Structure of PET Apparatus (Head Imaging State))

As shown in FIGS. 1 to 3, the PET apparatus 100 includes an imaging unit 1, a bed 2 on which the human body T as a target to be imaged is placed in a recumbent position, and a rotation mechanism 3 that rotates the imaging unit 1. As shown in FIG. 4, the PET apparatus 100 includes a controller 4 that controls the entire PET apparatus 100, and an image processor 5 that creates an image based on a detection signal from the imaging unit 1.

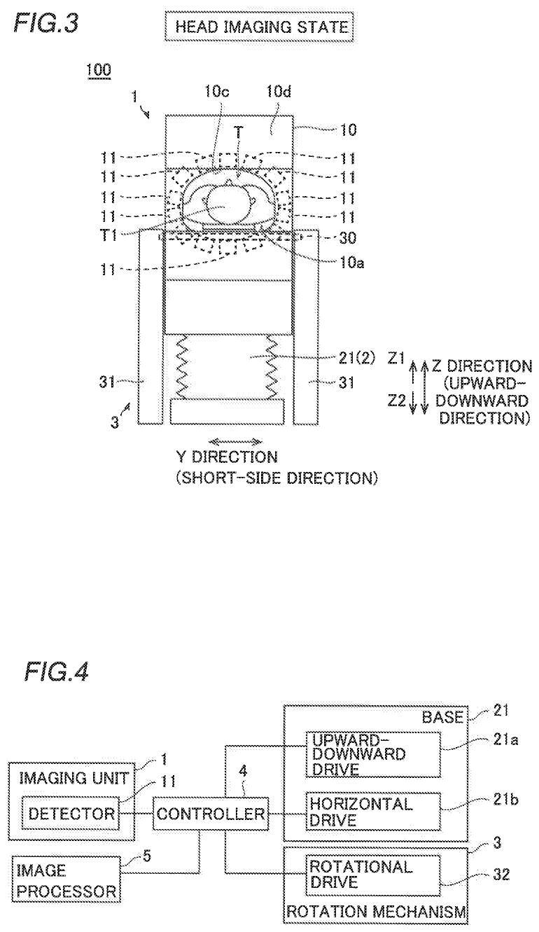

The imaging unit 1 is disposed on one side (X1 side) of the bed 2 in an X direction. Furthermore, as shown in FIG. 3, the imaging unit 1 includes a housing 10 and a plurality of (fourteen in the first embodiment) detectors 11 disposed inside the housing 10. The detector 11 is an example of an “imager” in the claims.

In the housing 10, as shown in FIG. 1, an imaging region 10a in which the target area to be imaged (the head T1 or the breast T2) of the human body T is placed is formed. The imaging region 10a includes a hole that passes through the housing 10. In the head imaging state, the imaging region 10a passes through the housing 10 in the X direction, in which the bed 2 extends, in a horizontal direction.

A head support 10b is provided on the X1 side (the side opposite to the bed 2) of the housing 10. In the head imaging state shown in FIGS. 1 to 3, the head support 10b extends downward from a corner of the housing 10 on the X1 side and on the lower side (Z2 side). Furthermore, the head support 10b has a plate shape. It should be noted that the head support 10b does not support the head T1 of the human body T in the head imaging state shown in FIGS. 1 to 3. The head support 10b is an example of a “support” in the claims.

The detectors 11 detect (collect) the gamma rays generated by the pair annihilation of the drug and transmit a detection signal to the image processor 5 via the controller 4. As shown in FIG. 1, in the head imaging state, each of the plurality of detectors 11 extends in the X direction over substantially the entire imaging region 10a that extends in the X direction.

As shown in FIG. 3, the plurality of detectors 11 surround the imaging region 10a inside the housing 10. In the head imaging state, the plurality of detectors 11 surround the head T1 of the supine human body T placed in the imaging region 10a within a vertical plane orthogonal to the X direction in which the imaging region 10a extends. Thus, the pair of gamma rays emitted in mutually opposite directions due to the pair annihilation of the drug can be reliably detected by a pair of detectors 11 that face each other. Consequently, the PET apparatus 100 acquires an internal image of the head T1 by detecting (imaging) the gamma rays with the detectors 11.

The bed 2 includes a top board 20 and a base 21 that supports the top board 20. As shown in FIG. 2, the top board 20 has a rectangular shape, which is long in a longitudinal direction (X direction) in a plan view. On the upper surface 20a of the top board 20, the human body T can lie in a recumbent position such as a prone position, a supine position, and a lateral recumbent position. As shown in FIG. 1, a headrest 22 is detachably attached to an end 20b of the top board 20 on the X1 side (imaging unit 1 side) in the longitudinal direction. The headrest 22 includes a support 22a that supports the head of the human body and a step 22b. The support 22a of the headrest 22 is located above (Z1 side) the upper surface 20a of the top board 20 by a predetermined height due to the step 22b. The headrest 22 is made of a material (carbon, for example) that does not absorb gamma rays.

The base 21 is disposed below the top board 20. The base 21 moves the top board 20 in an upward-downward direction (Z direction) and the longitudinal direction (X direction) while maintaining the horizontal state of the upper surface 20a of the top board 20. As shown in FIG. 4, the base 21 includes an upward-downward drive 21a and a horizontal drive 21b. The upward-downward drive 21a or the horizontal drive 21b of the base 21 is driven by the controller 4 such that the top board 20 is moved in the upward-downward direction or the X direction. Thus, in the head imaging state, the top board 20 can be moved in the X direction in a state in which the human body T is lying in the recumbent position (supine position) on the top board 20 such that the head T1 of the human body T is placed at a desired position in the imaging region 10a. In addition, the head T1 is imaged in a supine position by the PET apparatus 100, and thus the head T1 can be imaged in a stably fixed state. Thus, a clear image of the head T1 can be obtained.

As shown in FIGS. 1 to 3, the rotation mechanism 3 includes a rotary shaft 30 that rotates the imaging unit 1, a pair of support walls 31 that support the imaging unit 1 via the rotary shaft 30, and a rotational drive 32 (see FIG. 4) that rotates the rotary shaft 30. As shown in FIG. 1, the rotary shaft 30 is disposed proximally below an opening 10c of the imaging region 10a on the X1 side in the head imaging state. In addition, the rotary shaft 30 is disposed at substantially the same position (height) as that of the upper surface 20a of the top board 20 in the upward-downward direction. As shown in FIGS. 2 and 3, the rotary shaft 30 passes through the housing 10 in a short-side direction (Y direction) orthogonal to the longitudinal direction.

The pair of support walls 31 sandwich the imaging unit 1 in the short-side direction. The support walls 31 extend in the upward-downward direction.

In the first embodiment, the rotation mechanism 3 rotates the imaging unit 1 in the head imaging state shown in FIGS. 1 to 3 about the rotary shaft 30 by about 90 degrees in a clockwise direction (a rotational direction in which the head support 10b is away from the bed 2) R1 from a state in a side view in FIG. 1 so as to switch the imaging unit 1 to the breast imaging state shown in FIGS. 5 to 7. Furthermore, the rotation mechanism 3 rotates the imaging unit 1 in the breast imaging state shown in FIGS. 5 to 7 about the rotary shaft 30 by about 90 degrees in a counterclockwise direction (a rotational direction in which the head support 10b moves downward) R2 from a state in a side view in FIG. 5 so as to switch the imaging unit 1 to the head imaging state shown in FIGS. 1 to 3.

### (Structure of PET Apparatus (Breast Imaging State))

As a result, in the breast imaging state shown in FIGS. 5 to 7, the imaging unit 1 in the head imaging state shown in FIGS. 1 to 3 is rotated by about 90 degrees. Specifically, in the breast imaging state, the imaging region 10a of the imaging unit 1 extends in the upward-downward direction (Z direction) orthogonal to the upper surface 20a of the top board 20 of the bed 2, and passes through the housing 10, as shown in FIG. 5. Thus, in the PET apparatus 100, the breast T2 of the prone human body T can be placed in a hanging state within the imaging region 10a.

In the breast imaging state, as shown in FIG. 6, the plurality of detectors 11 of the imaging unit 1 surround the breast T2 of the prone human body T placed in the imaging region 10a within a horizontal plane orthogonal to the Z direction in which the imaging region 10a extends. Consequently, the PET apparatus 100 acquires an internal image of the breast T2 by detecting (imaging) the gamma rays with the detectors 11.

In the breast imaging state, a support surface 10d of the housing 10 of the imaging unit 1 is located slightly above (Z1 side) the upper surface 20a of the bed 2, as shown in FIG. 5. The support surface 10d is a surface located on the X1 side in the head imaging state shown in FIGS. 1 to 3. In the breast imaging state, the head support 10b extends from the support surface 10d toward the X1 side away from the bed 2. Thus, the head T1 and the upper portion of the prone human body T are supported by the head support 10b and the support surface 10d.

In the breast imaging state, each of the plurality of detectors 11 extends in the Z direction over substantially the entire imaging region 10a that extends in the Z direction. Furthermore, in the breast imaging state, the headrest 22 is detached from the top board 20. Thus, a portion of the breast T2 of the prone human body T corresponding to the height of the headrest 22 is prevented from failing to be placed within the imaging region 10a. In the breast imaging state, the rotary shaft 30 is disposed on the X1 side in the vicinity of the opening 10c of the imaging region 10a on the Z1 side. The rotation mechanism 3 can maintain the state (either the head imaging state or the breast imaging state) of the imaging unit 1 with a fixing mechanism (not shown).

### (Rotation Operation)

The rotation operation of the imaging unit 1 of the PET apparatus 100 according to the first embodiment is now specifically described with reference to FIGS. 1, 5, and 8 to 10.

In the case of the imaging unit 1 of the PET apparatus 100 in the head imaging state shown in FIG. 1, the horizontal drive 21b (see FIG. 4) is driven by the controller 4 (see FIG. 4) in a state in which the human body T is not placed on the bed 2, as shown in FIG. 8. Thus, the top board 20 of the bed 2 is moved toward the X2 side away from the imaging unit 1. At this time, the top board 20 is moved toward the X2 side at least to a position at which the housing 10 of the imaging unit 1 does not contact the top board 20 during rotation. Then, the headrest 22 is detached from the end 20b of the top board 20 on the X1 side.

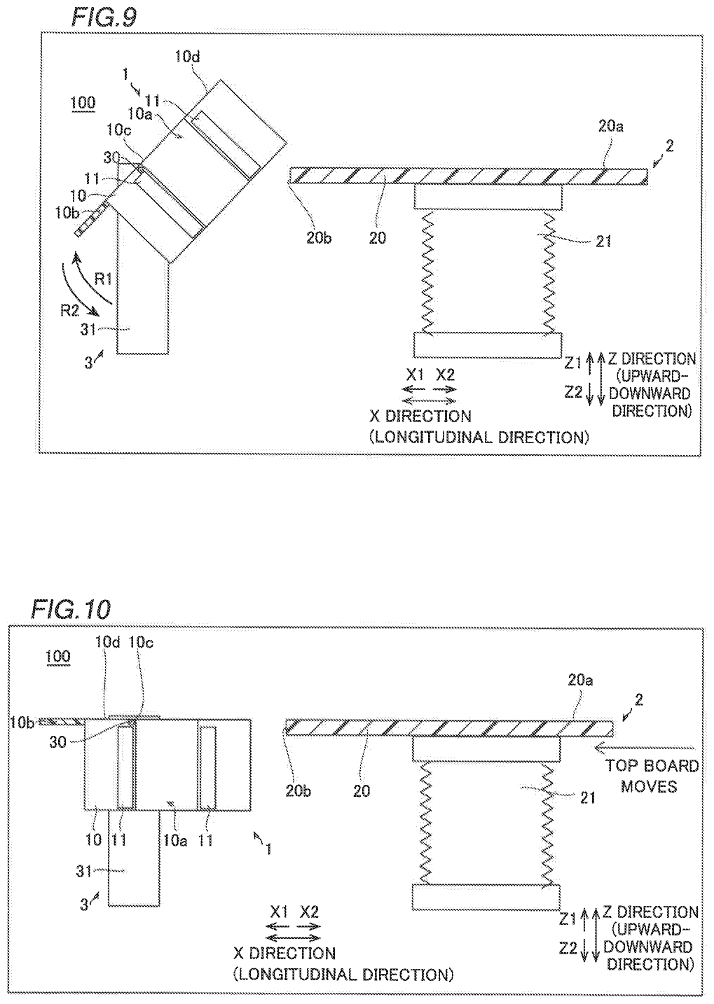

Thereafter, as shown in FIG. 9, the controller 4 drives the rotational drive 32 (see FIG. 4) to rotate the imaging unit 1 about the rotary shaft 30 by about 90 degrees in the R1 direction. Thus, the imaging region 10a is switched from a state of extending in the X direction to a state of extending in the Z direction. Finally, the controller 4 drives the horizontal drive 21b to move the top board 20 of the bed 2 to the X1 side toward the imaging unit 1. Thus, the imaging unit 1 is switched to the breast imaging state shown in FIG. 5. Thereafter, the top board 20 is appropriately moved in the X direction such that the head T1 of the supine human body T is placed at a desired position in the imaging region 10a.

When the imaging unit 1 of the PET apparatus 100 is switched from the breast imaging state shown in FIG. 5 to the head imaging state shown in FIG. 1, an operation opposite to the above switching is performed. That is, in the case of the imaging unit 1 in the breast imaging state shown in FIG. 5, the controller 4 drives the horizontal drive 21b in a state in which the human body T is not placed on the bed 2, as shown in FIG. 10. Thus, the top board 20 of the bed 2 is moved toward the X2 side away from the imaging unit 1. At this time, the top board 20 is moved toward the X2 side at least to a position at which the housing 10 of the imaging unit 1 does not contact the top board 20 during rotation.

Thereafter, as shown in FIG. 9, the controller 4 drives the rotational drive 32 to rotate the imaging unit 1 about the rotary shaft 30 by about 90 degrees in the R2 direction. Thus, the imaging region 10a is switched from a state of extending in the Z direction to a state of extending in the X direction. Then, the headrest 22 is attached to the end 20b of the top board 20 on the X1 side. Finally, the controller 4 drives the horizontal drive 21b to move the top board 20 of the bed 2 to the X1 side toward the imaging unit 1. Thus, the imaging unit 1 is switched to the head imaging state shown in FIG. 1. Thereafter, the human body T is placed in a prone position on the top board 20, the head support 10b, and the support surface 10d such that the breast T2 hangs downward within the imaging region 10a.

### Advantageous Effects of First Embodiment

According to the first embodiment, the following advantageous effects are achieved.

According to the first embodiment, as described above, the PET apparatus 100 includes the rotation mechanism 3 that rotates the imaging unit 1 to switch the imaging unit 1 to the head imaging state in which the head T1 of the human body T is imaged and the breast imaging state in which the breast T2 of the human body T different from the head T1 is imaged. Accordingly, the imaging unit 1 is rotated by the rotation mechanism 3 such that the imaging unit 1 can be switched to states in which different target areas to be imaged are imaged, and thus it is not necessary to provide a dedicated imaging unit for each area of the human body T. Consequently, in the PET apparatus 100, an increase in the number of components and the complex apparatus structure can be significantly reduced or prevented, and the installation cost of the PET apparatus 100 can be reduced.

According to the first embodiment, as described above, the detectors 11 surround the imaging region 10a in which the head T1 is placed in the head imaging state and the breast T2 is placed in the breast imaging state. Accordingly, the head T1 or the breast T2 placed in the imaging region 10a can be reliably imaged by the detectors 11 that surround the imaging region 10a.

According to the first embodiment, as described above, the rotary shaft 30 of the rotation mechanism 3 about which the imaging unit 1 is rotated extends, in the imaging unit 1, in the short-side direction (Y direction) orthogonal to the longitudinal direction (X direction) of the top board 20 of the bed 2. Here, when the rotary shaft 30 extends in the longitudinal direction, it is necessary to dispose the support walls 31 of the rotary shaft 30 disposed on the X1 side of the top board 20 so as not to interfere with the top board 20, and thus it is believed that the rotary shaft 30 and the rotation mechanism 3 are increased in size accordingly. Thus, as compared with the case in which the rotary shaft 30 extends in the longitudinal direction, an increase in the sizes of the rotary shaft 30 and the rotation mechanism 3 can be significantly reduced or prevented, and thus an increase in the size of the PET apparatus 100 can be further significantly reduced or prevented.

According to the first embodiment, as described above, the rotary shaft 30 is provided in the vicinity of the opening 10c of the imaging region 10a on the side (X1 side) opposite to the bed 2 in the imaging unit 1 in the head imaging state. Accordingly, approach of the rotation range of the imaging unit 1 to the bed 2 can be significantly reduced or prevented, and thus inhibition of rotation of the imaging unit 1 by the bed 2 can be significantly reduced or prevented. Furthermore, the rotary shaft 30 is provided in the vicinity of the opening 10c of the imaging region 10a such that the imaging region 10a can be located within the narrow rotation range around the rotary shaft 30, and thus spacing apart of the imaging region 10a from the bed 2 can be significantly reduced or prevented.

According to the first embodiment, as described above, the rotary shaft 30 is provided at substantially the same height as that of the upper surface 20a of the bed 2. Accordingly, the rotary shaft 30 is provided in the vicinity of the opening 10c of the imaging region 10a and at substantially the same height as that of the upper surface 20a of the bed 2 such that spacing apart of the imaging region 10a from the upper surface 20a of the bed 2 can be significantly reduced or prevented as compared with the case in which the rotary shaft 30 is provided at a height spaced apart from the upper surface 20a of the bed 2.

According to the first embodiment, as described above, the PET apparatus 100 includes the headrest 22 detachably attached to the end 20b of the bed 2 on the imaging unit 1 side (X1 side) and that supports the head T1 of the human body T. Accordingly, when the headrest 22 is attached to the end 20b of the bed 2 on the X1 side, the position of the headrest 22 is adjusted such that the head T1 of the recumbent (supine) human body T can be placed at an appropriate position in the imaging region 10a. Consequently, in the head imaging state, the head T1 of the recumbent human body T can be easily imaged by the detectors 11. Furthermore, the headrest 22 is detached from the end of the bed 2 on the X1 side such that it is possible to prevent the headrest 22 from interfering with rotation of the imaging unit 1.

According to the first embodiment, as described above, the head support 10b that supports the head T1 of the human body T extends, in the imaging unit 1, in the direction (X1 side) away from the bed 2 in the breast imaging state. Accordingly, in the breast imaging state in which the breast T2 located in the vicinity of the center of the human body T in the longitudinal direction is imaged, the head T1 of the recumbent (prone) human body T can be supported by the head support 10b that extends in the direction away from the bed 2. Consequently, the human body T can be securely kept in a recumbent position, and thus the breast T2 can be stably imaged.

According to the first embodiment, as described above, in the PET apparatus 100, the plurality of detectors 11 surround the head T1 of the human body T within the vertical plane in the head imaging state. Accordingly, the head T1 of the supine human body T can be stably and reliably imaged.

According to the first embodiment, as described above, in the PET apparatus 100, the plurality of detectors 11 surround the breast T2 of the human body T within the horizontal plane in the breast imaging state. Accordingly, the breast T2 of the human body T can hang downward in a prone position, and thus a wide range of the breast T2 of the human body T can be stably and reliably imaged. In FIG. 6, both the breasts T2 of the human body T are surrounded by the plurality of detectors 11, but only one breast may be surrounded by the plurality of detectors 11.

### Second Embodiment

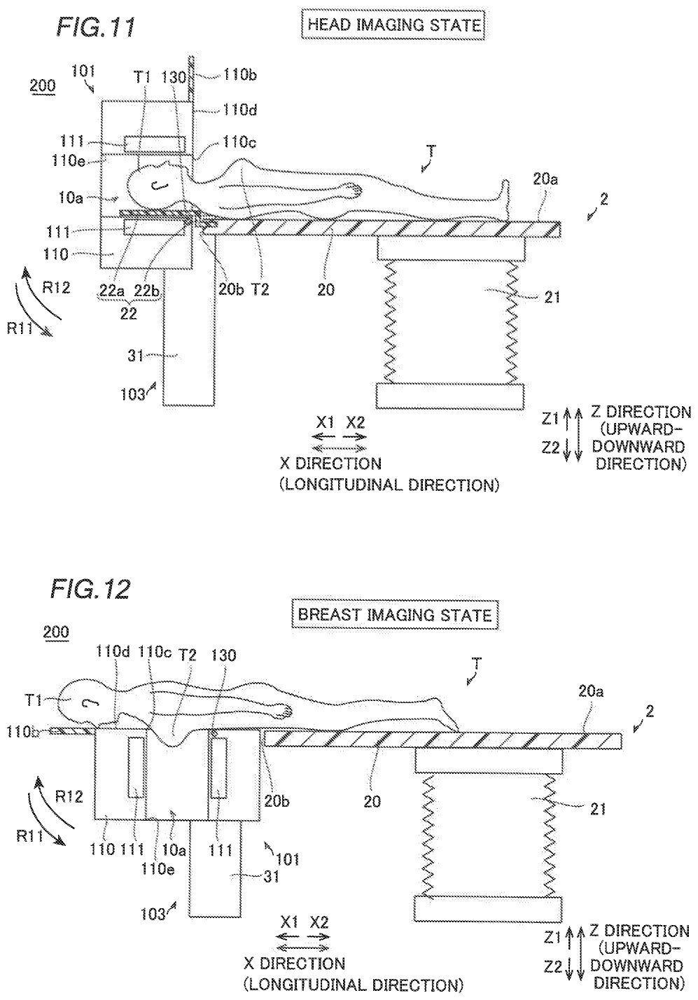

The structure of a PET apparatus 200 according to a second embodiment of the present invention is now described with reference to FIGS. 11 and 12. In the second embodiment, a rotary shaft 130 is provided on the bed 2 side in a head imaging state unlike the first embodiment. The same structures as those of the first embodiment are denoted by the same reference numerals, and description thereof is omitted. The PET apparatus 200 is an example of a “diagnostic imaging apparatus” in the claims.

### (Structure of PET Apparatus (Head Imaging State))

As shown in FIG. 11, the PET apparatus 200 includes an imaging unit 101, a bed 2, and a rotation mechanism 103 that rotates the imaging unit 101. The imaging unit 101 includes a housing 110 and a plurality of detectors 111 disposed inside the housing 110. The detectors 111 are examples of an “imager” in the claims.

In the head imaging state, a head support 110b is provided on the X2 side (the bed 2 side) of the housing 110. In the head imaging state shown in FIG. 11, the head support 110b extends upward from a corner of the housing 110 on the X2 side and the upper side (Z1 side). Furthermore, the head support 110b has a plate shape. It should be noted that the head support 110b does not support the head T1 of a human body T in the head imaging state. The head support 110b is an example of a “support” in the claims.

Unlike the detectors 11 according to the first embodiment, the plurality of detectors 111 extend in an X direction from the vicinity of an opening 110c on the X2 side, into which the head T1 is inserted, of an imaging region 10a that extends in the X direction to the vicinity of the center of the imaging region 10a in the head imaging state, but are not provided in the vicinity of an opening 110e of the imaging region 10a on the X1 side.

The rotation mechanism 103 includes the rotary shaft 130 in place of the rotary shaft 30 according to the first embodiment. In the head imaging state, the rotary shaft 130 is disposed proximally below the opening 110c of the imaging region 10a on the X2 side. In addition, the rotary shaft 130 is disposed at substantially the same position (height) as that of the upper surface 20a of a top board 20 in an upward-downward direction.

In the second embodiment, the rotary mechanism 103 rotates the imaging unit 101 in the head imaging state shown in FIG. 11 about the rotary shaft 130 by about 90 degrees in a counterclockwise direction (a rotational direction in which the head support 110b is away from the bed 2) R11 from a state in a side view in FIG. 11 so as to switch the imaging unit 101 to a breast imaging state shown in FIG. 12. Furthermore, the rotary mechanism 103 rotates the imaging unit 101 in the breast imaging state shown in FIG. 12 about the rotary shaft 130 by about 90 degrees in a clockwise direction (a rotational direction in which the head support 110b moves upward) R12 from a state in a side view in FIG. 12 so as to switch the imaging unit 101 to the head imaging state shown in FIG. 11. That is, in the PET apparatus 200 according to the second embodiment, the state of the imaging unit 101 is switched as the imaging unit 101 rotates in a direction opposite to the rotational direction of the imaging unit 1 according to the first embodiment.

### (Structure of PET Apparatus (Breast Imaging State))

In the breast imaging state shown in FIG. 12, the imaging unit 101 in the head imaging state shown in FIG. 11 is rotated by about 90 degrees. Specifically, in the breast imaging state, the detectors 111 extend in a Z direction from the vicinity of the opening 110c on the Z1 side, into which a breast T2 is inserted, of the imaging region 10a that extends in the Z direction to the vicinity of the center of the imaging region 10a, unlike the detectors 11 according to the first embodiment. Consequently, in order to image the head T1 or the breast T2, the detectors 111 may not be disposed in the vicinity of the opening 110e into which the head T1 or the breast T2 is not inserted as long as the detectors 111 are disposed in the vicinity of the opening 110c into which the head T1 or the breast T2 is inserted. Thus, the detectors 111 can be downsized, and thus it is possible to reduce the cost of the detectors 111 and to increase the degree of freedom of arrangement of the remaining members in the imaging unit 101.

In the second embodiment, the PET apparatus 200 has the same positional relationship as that of the PET apparatus 100 in the breast imaging state according to the first embodiment, except for the detectors 111, when the imaging unit 101 in the head imaging state rotates in the direction R11 opposite to the rotational direction R1 of the imaging unit 1 according to the first embodiment. The remaining structures of the PET apparatus 200 according to the second embodiment are similar to those of the PET apparatus 100 according to the first embodiment. Furthermore, the rotation operation of the PET apparatus 200 according to the second embodiment is the same as that of the PET apparatus 100 according to the first embodiment except that the rotational direction at the time of switching is reversed, and thus description thereof is omitted.

### Advantageous Effects of Second Embodiment

According to the second embodiment, the following advantageous effects are achieved.

According to the second embodiment, as described above, the PET apparatus 200 includes the rotary mechanism 103 that rotates the imaging unit 101 so as to switch the imaging unit 101 to the head imaging state in which the head T1 of the human body T is imaged and the breast imaging state in which the breast T2 of the human body T different from the head T1 is imaged. Accordingly, similarly to the first embodiment, in the PET apparatus 200, an increase in the number of components and the complex apparatus structure can be significantly reduced or prevented.

According to the second embodiment, as described above, the rotary shaft 130 is provided in the vicinity of the opening 110c of the imaging region 10a on the bed 2 side (X2 side) in the imaging unit 101 in the head imaging state. Accordingly, protrusion of support walls 31 of the rotation mechanism 103 to the side (X1 side) of the PET apparatus 200 opposite to the bed 2 can be significantly reduced or prevented, and thus an increase in the size of the PET apparatus 200 can be significantly reduced or prevented. Furthermore, the rotary shaft 130 is provided in the vicinity of the opening 110c of the imaging region 10a such that the imaging region 10a can be located within the narrow rotation range around the rotary shaft 130, and thus spacing apart of the imaging region 10a from the bed 2 can be significantly reduced or prevented.

According to the second embodiment, as described above, the detectors 111 extend in the Z direction from the vicinity of the opening 110c on the Z1 side, into which the breast T2 is inserted, of the imaging region 10a that extends in the Z direction to the vicinity of the center of the imaging region 10a in the breast imaging state. Accordingly, the detectors 111 can be downsized, and thus it is possible to reduce the cost of the detectors 111 and to increase the degree of freedom of arrangement of the remaining members in the imaging unit 101. The remaining advantageous effects of the second embodiment are similar to those of the first embodiment.

### Third Embodiment

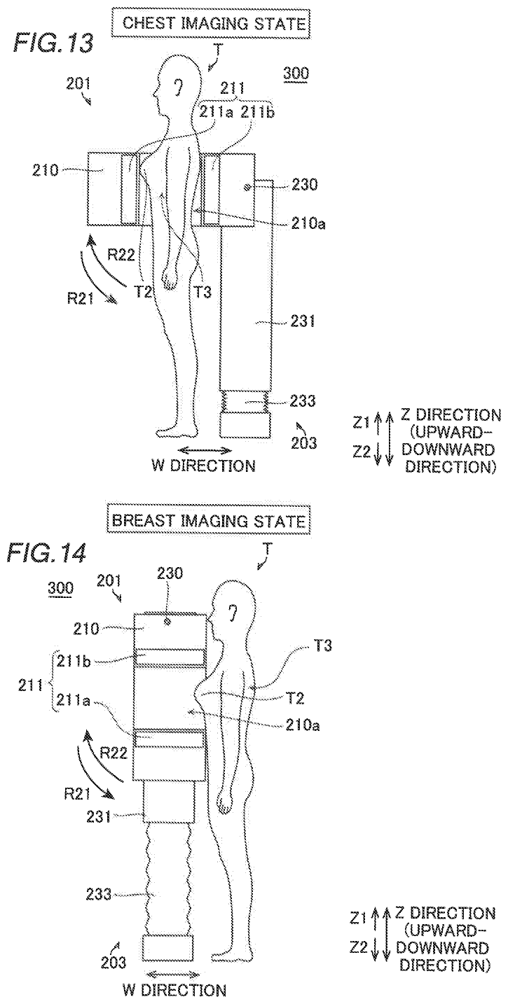

The structure of an X-ray imaging apparatus 300 according to a third embodiment of the present invention is now described with reference to FIGS. 13 and 14. In the third embodiment, the X-ray imaging apparatus 300 images a human body T in a standing position unlike the first embodiment. The X-ray imaging apparatus 300 is an example of a “diagnosis imaging apparatus” in the claims.

### (Outline of X-ray Imaging Apparatus)

The X-ray imaging apparatus 300 is an apparatus that captures an image (simple X-ray image) inside the human body T, utilizing the fact that the degree of X-ray absorption in the body is different. The image formed in the X-ray imaging apparatus 300 is used for image diagnosis.

In the third embodiment, the X-ray imaging apparatus 300 can switch between a chest imaging state in which the chest T3 of the standing human body T is imaged and a breast imaging state in which the breast T2 of the standing human body T is imaged. In the third embodiment, the structure of the X-ray imaging apparatus 300 in the chest imaging state shown in FIG. 13 is described first, and then points of the breast imaging state of the X-ray imaging apparatus 300 shown in FIG. 14 different from the chest imaging state are described. The chest T3 and the chest imaging state are examples of a “first target area to be imaged” and a “first state” in the claims, respectively.

### (Structure of X-ray Imaging Apparatus (Chest Imaging State))

As shown in FIG. 13, the X-ray imaging apparatus 300 includes an imaging unit 201 and a rotation mechanism 203 that rotates the imaging unit 201. The X-ray imaging apparatus 300 further includes a controller (not shown) that totally controls the X-ray imaging apparatus 300 and an image processor (not shown) that creates an image based on a detection signal from the imaging unit 201.

The imaging unit 201 includes a housing 210 and an imager 211 disposed inside the housing 210. At the center of the housing 210, an imaging region 210a in which the target area to be imaged (the chest T3 or the breast T2) of the human body T is placed is formed. The imaging region 210a includes a hole that passes through the housing 210. In the chest imaging state shown in FIG. 13, the imaging region 210a passes through the housing 210 so as to extend in an upward-downward direction (Z direction).

The imager 211 includes an X-ray source 211a that radiates X-rays in a predetermined direction (W direction) in which the imaging region 210a is located in the chest imaging state, and an X-ray detector 211b that faces the X-ray source 211a in the W direction and detects the X-rays. In other words, the X-ray source 211a and the X-ray detector 211b sandwich the imaging region 210a in the W direction. Consequently, the X-ray imaging apparatus 300 acquires an internal image of the chest T3 placed in the imaging region 210a.

The rotation mechanism 203 includes a rotary shaft 230 that rotates the imaging unit 201, support walls 231 that support the imaging unit 201 via the rotary shaft 230, and a lifting mechanism 233 that moves the support walls 231 in the upward-downward direction (Z direction). The lifting mechanism 233 is disposed below (Z2 side) the support walls 231. Furthermore, the imaging unit 201 is rotated about the rotary shaft 230 by a rotational drive (not shown).

In the third embodiment, the rotation mechanism 203 rotates the imaging unit 201 in the chest imaging state shown in FIG. 13 about the rotary shaft 230 by about 90 degrees in a counterclockwise direction R21 from a state in a side view in FIG. 13 so as to switch the imaging unit 201 to the breast imaging state shown in FIG. 14. Furthermore, the rotation mechanism 203 rotates the imaging unit 201 in the breast imaging state shown in FIG. 14 about the rotary shaft 230 by about 90 degrees in a clockwise direction R22 from a state in a side view in FIG. 14 so as to switch the imaging unit 201 to the chest imaging state shown in FIG. 13.

### (Structure of X-ray Imaging Apparatus (Breast Imaging State))

In the breast imaging state shown in FIG. 14, the imaging unit 201 in the chest imaging state shown in FIG. 13 is rotated by about 90 degrees. Specifically, in the breast imaging state, the imaging region 210a of the imaging unit 201 passes through the housing 210 so as to extend in the W direction. In the breast imaging state, the X-ray source 211a and the X-ray detector 211b sandwich the imaging region 210a in the upward-downward direction (Z direction). Thus, the imager 211 images the breast T2 of the standing human body T placed in the imaging region 210a while sandwiching the breast T2 in the upward-downward direction.

### (Rotation Operation)

The rotation operation of the imaging unit 201 of the X-ray imaging apparatus 300 according to the third embodiment is now specifically described with reference to FIGS. 13 and 14.

The imaging unit 201 of the X-ray imaging apparatus 300 in the chest imaging state shown in FIG. 13 is rotated about the rotary shaft 230 by about 90 degrees in the R21 direction. Thus, the imaging region 210a is switched from a state of extending in the Z direction to a state of extending in the W direction. Finally, the positions of the support walls 231 in the upward-downward direction are adjusted by the lifting mechanism 233 such that in the upward-downward direction, the height of the imaging region 210a is matched to the height of the breast T2 of the standing human body T. Thus, the imaging unit 201 of the X-ray imaging apparatus 300 is switched to the breast imaging state shown in FIG. 14.

When the imaging unit 201 of the X-ray imaging apparatus 300 is switched from the breast imaging state shown in FIG. 14 to the chest imaging state shown in FIG. 13, an operation opposite to the above switching is performed. That is, in the X-ray imaging apparatus 300 in the breast imaging state shown in FIG. 14, the imaging unit 201 is rotated about the rotary shaft 230 by about 90 degrees in the R22 direction. Thus, the imaging region 210a is switched from a state of extending in the W direction to a state of extending in the Z direction. Finally, the positions of the support walls 231 in the upward-downward direction are adjusted by the lifting mechanism 233 such that in the upward-downward direction, the height of the imaging region 210a is matched to a height at which the chest T3 of the standing human body T is imaged. Thus, the X-ray imaging apparatus 300 is switched to the chest imaging state shown in FIG. 13.

### Advantageous Effects of Third Embodiment

According to the third embodiment, the following advantageous effects are achieved.

According to the third embodiment, as described above, the X-ray imaging apparatus 300 includes the rotation mechanism 203 that rotates the imaging unit 201 to switch the imaging unit 201 to the chest imaging state in which the chest T3 of the human body T is imaged and the breast imaging state in which the breast T2 of the human body T different from the chest T3 is imaged. Accordingly, similarly to the first embodiment, in the X-ray imaging apparatus 300, an increase in the number of components and the complex apparatus structure can be significantly reduced or prevented.

### Modified Examples

The embodiments disclosed this time must be considered as illustrative in all points and not restrictive. The scope of the present invention is not shown by the above description of the embodiments but by the scope of claims for patent, and all modifications (modified examples) within the meaning and scope equivalent to the scope of claims for patent are further included.

For example, while the PET apparatuses 100 and 200 are respectively shown as an example of the “diagnostic imaging apparatus” according to the present invention in the aforementioned first and second embodiments, and the X-ray imaging apparatus 300 is shown as an example of the “diagnostic imaging apparatus” according to the present invention in the aforementioned third embodiment, the present invention is not restricted to this. The structure according to the present invention may alternatively be applied to an X-ray CT (Computed Tomography) apparatus, an optical CT apparatus, an ultrasonic CT apparatus, an MRI (Magnetic Resonance Imaging) apparatus, etc. used for image diagnosis as the diagnostic imaging apparatus according to the present invention.

While the imaging units 1 and 101 are switched to the head imaging state and the breast imaging state so as to respectively image the head T1 (first target area to be imaged) and the breast T2 (second target area to be imaged) of the human body T in the aforementioned first and second embodiments, and the imaging unit 201 is switched to the chest imaging state and the breast imaging state so as to respectively image the chest T3 (first target area to be imaged) and the breast T2 (second target area to be imaged) of the human body T in the aforementioned third embodiment, the present invention is not restricted to this. According to the present invention, a target area to be imaged of the human body other than the head, the breast, and the chest may be able to be imaged as long as different target areas to be imaged of the human body can be imaged. Furthermore, the target to be imaged is not restricted to the human body. For example, the body of an animal other than a human being may alternatively be a target to be imaged.

While the headrest 22 and the head supports 10b and 110b are provided in the PET apparatuses (diagnostic imaging apparatuses) 100 and 200 in the aforementioned first and second embodiments, the present invention is not restricted to this. According to the present invention, the headrest or the head support may not be provided in the diagnostic imaging apparatus.

While the imaging unit 1 (101, 201) is rotated about the rotary shaft 30 (130, 230) by about 90 degrees to be switched to the first state and the second state in each of the aforementioned first to third embodiments, the present invention is not restricted to this. According to the present invention, the rotation angle may alternatively be an angle other than about 90 degrees. For example, in the third embodiment, when the breast is imaged in a tilted state, the imaging unit may be rotated by a rotation angle of less than about 90 degrees to be switched to the first state (chest imaging state) and the second state (breast imaging state).

Furthermore, the inner diameters or the shapes of the imaging regions 10a and 210a may alternatively change according to the target area to be imaged in the aforementioned first to third embodiments. For example, in the first and second embodiments, in the diagnostic imaging apparatus (PET apparatus 100 (200)), the width of the imaging region 10a in the upward-downward direction in the imaging unit 1 (101) may be increased in the head imaging state, and the width of the imaging region 10a in the horizontal direction in the imaging unit 1 (101) may be decreased in the breast imaging state. The diagnostic imaging apparatus is configured as described above such that it is possible to image the target area to be imaged (head or breast) more easily and in more detail.

While the rotary shafts 30 and 130 are provided at substantially the same height as that of the upper surface 20a of the bed 2 in the aforementioned first and second embodiments, the present invention is not restricted to this. According to the present invention, the position of the rotary shaft is not particularly restricted as long as the imaging unit is rotatable.

What is claimed is:

1. A diagnostic imaging apparatus comprising:
an imaging unit including an imager that images a target to be imaged; and
a rotation mechanism that rotates the imaging unit to switch the imaging unit to a first state in which a first target area to be imaged of the target is imaged and a second state in which a second target area to be imaged of the target different from the first target area to be imaged is imaged.

2. The diagnostic imaging apparatus according to claim 1, wherein
the imaging unit further includes an imaging region in which the first target area to be imaged is placed in the first state and the second target area to be imaged is placed in the second state, and
the imager surrounds the imaging region.

3. The diagnostic imaging apparatus according to claim 1, further comprising a bed on which a human body as the target to be imaged is placed in a recumbent position, wherein
the rotation mechanism includes a rotary shaft that extends, in the imaging unit, in a short-side direction orthogonal to a longitudinal direction of the bed and about which the imaging unit is rotated.

4. The diagnostic imaging apparatus according to claim 3, wherein
the imaging unit further includes an imaging region in which a head of the human body in the recumbent position as the first target area to be imaged is placed in the first state and a breast of the human body in the recumbent position as the second target area to be imaged is placed in the second state, and
the rotary shaft is provided in a vicinity of an opening of the imaging region on a side opposite to the bed in the imaging unit in the first state.

5. The diagnostic imaging apparatus according to claim 3, wherein
the imaging unit further includes an imaging region in which a head of the human body in the recumbent position as the first target area to be imaged is placed in the first state and a breast of the human body in the recumbent position as the second target area to be imaged is placed in the second state, and
the rotary shaft is provided in a vicinity of an opening of the imaging region on the bed side in the imaging unit in the first state.

6. The diagnostic imaging apparatus according to claim 4, wherein the rotary shaft is provided at substantially a same height as that of an upper surface of the bed.

7. The diagnostic imaging apparatus according to claim 3, further comprising a headrest detachably attached to an end of the bed on the imaging unit side and that supports a head of the human body.

8. The diagnostic imaging apparatus according to claim 3, wherein the imaging unit includes a support that extends in a direction away from the bed in the second state and supports the human body.

9. The diagnostic imaging apparatus according to claim 1, wherein
the first target area to be imaged is a head of the human body in a supine position, and the imager surrounds the head of the human body within a vertical plane in the first state, and
the second target area to be imaged is a breast of the human body in a prone position, and the imager surrounds the breast of the human body within a horizontal plane in the second state.
